(19) United States
(12) Patent Application Publication
(10) Pub. No.: US 2007/0099869 A1
(43) Pub. Date: May 3, 2007

(54) Association product of α-glycosyl α,α-trehalose with ionic metal compound

(75) Inventors: Kazuyuki Oku, Okayama (JP); Michio Kubota, Okayama (JP); Shigeharu Fukuda, Okayama (JP); Toshio Miyake, Okayama (JP)

Correspondence Address: BROWDY AND NEIMARK, P.L.L.C.;624 NINTH STREET, NW, SUITE 300, WASHINGTON, DC, 20001-5303, US

(73) Assignee: KABUSHIKI KAISHA HAYASHIBARA SEIBUTSU KAGAKU KENKYUJO, OKAYAMA-SHI (JP)

(21) Appl. No.: 10/559,812

(22) PCT Filed: Jun. 6, 2004
(86) PCT No.: PCT/JP04/08105

§ 371 Date: Dec. 8, 2005

(30) Foreign Application Priority Data
Jun. 10, 2003 (JP) 164886/2003

(51) Int. Cl. A61K 31/715

(52) U.S. Cl. 514/61

(57) ABSTRACT

The present invention has an object to provide a composition of metal ion compound whose inherent and unsatisfactory properties for its industrial application such as deliquescence, reducing power, oxidizing power, low solubility in water, etc., have been improved; and their preparations and uses. The present invention solves the above object by providing an associated complex of α-glycosyl α,α-trehalose and a metal ion compound, and its preparations and uses.

### TECHNICAL FIELD

The present invention relates to a novel associated complex of a saccharide and a metal ion compound, and more particularly, to an associated complex of α-glycosyl α,α-trehalose and a metal ion compound.

### BACKGROUND ART

Metal elements such as sodium, potassium, calcium, magnesium, iron, copper, zinc, nickel, etc., are not required in a large amount for living bodies in comparison with carbon, oxygen, hydrogen, nitrogen, etc. However, the metal elements are essential for keeping biological functions in normal levels. Living bodies usually take such a metal element in the form of a compound comprising an ionic metal element (metal ion compound) such as salts. Then, a metal ion compound exhibits its functions in the bodies.

Magnesium and calcium are known to be minerals which involve many enzymatic reactions in human bodies, and are required in larger amounts. Lack of magnesium and calcium causes bone-thinning osteoporosis and osteomalacia because these metal elements are to be present in relatively large amounts in the bone of living bodies. Recently, a deficiency of magnesium was recognized as a cause of diseases such as diabetes and hypertension.

Magnesium is one of essential minerals in plants; it is generally supplied to plants as a fertilizer with a nitrogen, phosphorus, and potassium, in a liquid or solid form, and used for their growth. Lack of magnesium is known to be a cause of deficiency diseases in plants.

While, in the field of food industries, there is a case where metal ion compounds may exhibit disadvantages such as deliquescence, reducing power, oxidizing power, low solubility in water, etc. in the production and preservation of foods or their materials. These properties of metal ion compounds have been recognized as inherent properties which can not be improved. Also, there is a case that metal ion compounds such as salts may induce unpleasant tastes when orally administrated depending on the dose. Therefore, various studies have been made to reduce such an unpleasant taste. Recently, International Publication No. WO 03/016325, applied for by the same applicant as the present invention, disclosed that the above inherent disadvantages of metal ion compounds can be decreased by forming various metal ion compounds into associated complexes with a non-reducing disaccharide, α,α-trehalose, or a sugar alcohol, maltitol. The development for a variety of saccharides except for α,α-trehalose and maltitol, having a function as found in α,α-trehalose and maltitol, has been desired. However, no saccharide has been discovered.

An object of the present invention is to provide a composition comprising a metal ion compound whose inherent and unsatisfactory properties for its industrial application for foods, such as deliquescence, reducing power, oxidizing power, low solubility in water, etc., have been improved; and its preparation and uses.

### DISCLOSURE OF INVENTION

In order to solve the above object, the present inventors have extensively studied based on their knowledge of saccharides and uses thereof. At first, the changes of inherent properties of metal ion compounds were extensively investigated by combining them with various saccharides. As a result, it was revealed that non-reducing saccharides, α-glycosyl α,α-trehaloses, exhibited functions of improving deliquescence of metal ion compounds, increasing their solubility in water, and inhibiting their oxidative/reductive actions, as the cases of combining α,α-trehalose and maltitol with metal ion compounds to form associated complexes. Successively, in order to investigate the mechanism of exhibiting the above functions, interactions between α-glycosyl α,α-trehalose and metal ion compounds were analyzed in detail on their molecular level. As a result, it was revealed that the α-glycosyl moiety of α-glycosyl α,α-trehalose involved in the formation of associated complexes with metal ion compounds as well as the α,α-trehalose moiety, and that the resulting associated complexes showed the different properties from those of intact metal compounds. From the results described above, it was revealed that associated complexes, which are obtainable by allowing α-glycosyl α,α-trehalose to coexist with metal ion compounds, exhibited great merits in food industries. The present invention has been accomplished on the basis of the above knowledge.

The present invention solves the above object by providing associated complexes of α-maltosyl α,α-trehalose and either metal ion compounds, and their preparations and uses.

### BRIEF DESCRIPTION OF DRAWINGS

FIG. 1 shows an X-ray diffraction pattern of a crystalline associated complex of α-maltosyl α,α-trehalose and calcium chloride.

FIG. 2 shows an X-ray diffraction pattern of a crystalline calcium chloride dihydrate.

### BEST MODE FOR CARRYING OUT THE INVENTION

The present invention relates to an associated complex of α-maltosyl α,α-trehalose and a metal ion compound, its preparation and uses. The term “α-glycosyl α,α-trehalose” as referred to as in the present invention means non-reducing saccharides where a α-glycosyl residue constructed with one or more glucose residues are bound via the α-1,4 linkage with either of glucosyl residues of α,α-trehalose, a disaccharide constructed with two glucose molecules, for example, α-glucosyl α,α-trehalose, α-maltosyl α,α-trehalose, α-maltotriosyl α,α-trehalose, α-maltotetraosyl α,α-trehalose, α-maltopentaosyl α,α-trehalose, α-maltohexaosyl α,α-trehalose, etc. In the present invention, α-glycosyl α,α-trehaloses are not restricted by their purities and forms as far as they each form associated complexes with metal ion compounds described later. α-Glycosyl α,α-trehalose, usable in the present invention, can be prepared by the conventional method disclosed in Japanese Patent Kokai No. 143876/95. While, commercially available ones comprising α-maltosyl α,α-trehalose can be arbitrarily used. For example, “HALLODEX®, a syrup comprising about 4% of α-glucosyl α,α-trehalose, about 52% of α-maltosyl α,α-trehalose, and about 1% of α-maltotriosyl α,α-trehalose, on a dry solid basis, commercialized by Hayashibara Shoji, Inc., Okayama, Japan, can be advantageously used.

The term “metal ion compound” as referred to as in the present invention means a compound having a metal ion as a cation in compounds having an ionic bond between cation and anion, and includes a salt, alkali, or complex. In the present invention, any metal ion compound, which can form an associated complex with α-glycosyl α,α-trehalose, can be advantageously used. For example, a metal ion compounds comprises one or more metal ions having monovalent (univalent) or more ionic charges as cation, particularly, one or more metal ions of the groups 1 to 16 of the periodic table of the elements, more particularly, one or more metal ions selected from lithium, sodium, potassium, rubidium, etc., of the group 1; beryllium, magnesium, calcium, strontium, etc. of the group 2; scandium, yttrium, etc. of the group 3; titanium, zirconium, hafnium, etc., of the group 4; vanadium, niobium, tantalum, etc., of the group 5; chromium, molybdenum, tungsten, etc., of the group 6; manganese, technetium, rhenium, etc., of the group 7; iron, ruthenium, etc., of the group 8; cobalt, rhodium, etc., of the group 9; nickel, palladium, etc., of the group 10; copper, silver, etc., of the group 11; zinc, etc., of the group 12; aluminum, gallium, etc., of the group 13; germanium, etc., of the group 14; antimony, etc., of the group 15; and polonium, etc., of the group 16. In those compounds, metal ion compounds, comprising one or more metal ions selected from the group of alkali earth metal ions such as calcium, magnesium, strontium ions, etc.; metal ions belonging to transition elements such as ferrous, copper, nickel, manganese, zinc ions, etc.; and alkali metal ions such as sodium, potassium, etc., have a relatively remarkable usefulness as described later in detail as associated complexes. Since metal compounds, comprising metal ions having divalent or more charges, are more useful, they are especially useful in the present invention. One or more anions selected from halogen ions such as fluorine ion, chlorine ion, and bromine ion (except in the case of metal ions having calcium ion as counter ion), inorganic anions such as sulfate ion, sulfite ion, mono-hydrogen sulfate ion, thiosulfate ion, carbonate ion, bicarbonate ion, nitrate ion, phosphate ion, mono-hydrogen phosphate ion, di-hydrogen phosphate ion, chlorite ion, hydroxide ion, and ammonium ion, and organic anions such as acetate ion, lactate ion, citrate ion, fumaric ion, and malate ion, can be used for an ions of metal ion compounds in the present invention. Since metal ion compounds having inorganic anions are relatively useful as described below, metal ion compounds comprising inorganic anions can be advantageously used. Regarding the application of metal ion compounds of the present invention to living bodies, physiologically acceptable metal ion compounds are desirably used. Hereinafter, the term “metal ion compounds” means all metal ion compounds.

The term “associated complex” as referred to as in the present invention means a substance associating α-glycosyl α,α-trehalose and a metal ion compound via a direct interaction. Such associated complexes in the present invention are substantially constructed by α-glycosyl α,α-trehalose and metal ion compounds. The term “direct interaction” as referred to as in the present invention means hydrogen bond, van der Waals force, ionic bond, or coordinate bond, and includes those in solid, gas, liquid or paste state. Also, the term “substantially constructed by α-glycosyl α,α-trehalose and a metal ion compound”, as referred to as in the present invention means an associated complex which is usually constructed by α-glycosyl α,α-trehalose and a metal compound, and depending on circumstances, further means those which comprise other molecules such as bound water as constituents. Metal ion compounds in the above associated complexes are usually being associated with α-glycosyl α,α-trehalose in a neutralizing form (for example, salts, etc.) of metal ion and its counter ion. Depending on circumstances, metal ions can associated with α-glycosyl α,α-trehalose, and the counter ion bounds to neutralize the resulting associated complex. The associated complexes of the present invention can be identified as follows: An associated complex formed in solution can be identified by nuclear magnetic resonance (NMR) method described, for example, in “Jikken-Kagaku-Koza (Course of experimental chemistry) 5”, edited by The Chemical Society of Japan, published by Maruzen Co., Ltd., pp. 221-224 (1991). An associated complex can be identified by the steps of analyzing a solution comprising an associated complex of the present invention and a solution comprising only α-glycosyl α,α-trehalose by NMR, comparing the relaxation time of constituent atoms, and detecting the phenomenon that atoms of the associated complex show shorter relaxation times. Further, such an associated complex can be identified by the fact that one or more signals assigned with chemical shifts (ppm) show signals with different chemical shifts in comparison with the corresponding signals observed in the case of using α-glycosyl α,α-trehalose alone. Furthermore, the associated complex can be also identified by crystallizing it in solutions, isolating the resulting crystals, and analyzing the crystallographic structure. Specifically, the associated complexes of the present invention can be identified by the steps of analyzing their X-ray diffraction patterns, and comparing them with those of crystalline metal ion compound alone; and confirming the fact that X-ray diffraction patterns of the associated complexes are not agreed with any of those obtained from the metal ion compounds, and combination patterns thereof. The associated complexes of the present invention, described above, comprise α-glycosyl α,α-trehalose and metal ion compounds (or metal ion) in molar ratios of, usually, 1:0.5 to 1:5, desirably, 1:1 to 1:4. As in the case of crystalline associated complex of α-maltosyl α,α-trehalose and calcium chloride, which is described later in detail in Experiments, the associated complex may give a prescribed molar ratio of α-glycosyl α,α-trehalose and the metal ion of about 1:1.

The associated complex of the present invention can be formed by mixing α-glycosyl α,α-trehalose with a metal ion compound. The mixing can be done by contacting α-glycosyl α,α-trehalose with a metal ion compound. Usually, it is preferable to mix them using the same solvent under a condition which the both components are dissolved. Water, ethanol, methanol, acetonitrile, dimethylsulfoxide, dimethylformamide, and acetic acid can be used as the solvent. In the case of preparing the associated complex to apply to living bodies for using in the fields of foods, cosmetics, and pharmaceuticals, physiologically acceptable solvents such as water and ethanol can be desirably used. In the case of using a hydrous form of metal ion compounds or inherently deliquescent metal ion compounds such as calcium chloride, it is possible to form desired associated complexes by mixing them into solid forms. Although the proportion of α-glycosyl α,α-trehalose to a metal ion compound depends on the kinds of metal ion compounds, the molar ratio of a metal ion compound to α-glycosyl α,α-trehalose can be preferably set to in the range of, usually, 0.01 or higher but 100 or lower, desirably, 0.1 or higher but 10 or lower. As in the case of an associated complex of α-maltosyl α,α-trehalose and calcium chloride, which are described later in detail in Experiments; the complex can be obtained efficiently by mixing α-glycosyl α,α-trehalose and a metal ion compound in prescribed molar ratio such as about one.

The associated complex of α-glycosyl α,α-trehalose and metal ion compound, formed as described above, can be used intact, for example, as solution forms; and they can be also used as an isolated form. For example, extraction, filtration, concentration, centrifugation, dialysis, precipitation, crystallization, hydrophobic chromatography, gel-filtration chromatography, and affinity chromatography can be used as the method for isolating the associated complex.

The associated complex, formed as described above, or fractions containing the same can be collected by methods such as crystallization, precipitation, concentration, and drying (including a spray drying, drying in vacuo, and freeze-drying). Depending on the kinds of metal ion compounds, the associated complex, obtainable by the above methods, has excellent properties in comparison with conventional preparations of a metal ion compound as follows:

(1) Reduction of Deliquescence

Alkaline earth metal halides including calcium chloride have deliquescent properties. The deliquescent properties of the metal ion compounds are remarkably reduced by forming associated complexes with α-glycosyl α,α-trehalose. Therefore, there is a feature of having a satisfactory handleability in the associated complexes of the present invention, which comprise metal ion compounds having an inherent deliquescence. Such properties can be advantageously applied to produce deliquescence-inhibiting agents.

(2) Inhibition of Forming Hardly Soluble or Insoluble Metal Ion Compounds

In some cases, metal ions form salts having a low solubility in water depending on the kind of counter ions as in the case of calcium phosphate. When a counter ion, which forms hardly soluble or insoluble salts with such metal ions, is added to a solution comprising such metal ions, a substance (salt) having a low solubility is rapidly formed and precipitated. The formation of such a hardly soluble or insoluble salt can be inhibited by forming associated complex of a compound comprising such a metal ion and α-glycosyl α,α-trehalose before the formation of a hardly soluble or insoluble salt. Therefore, associated complex of a compound, comprising a metal ion which is inherently capable of forming a hardly soluble or insoluble salt, and α-glycosyl α,α-trehalose can be used as a preparation whose precipitation or clouding in water is inhibited. Such properties can be applied to produce a precipitation-inhibiting agent for hardly soluble or insoluble salts.

(3) Improved Solubility in Water

Metal ion compounds, which are in an associated complex form with α-glycosyl α,α-trehalose, have, in many cases, a higher solubility in water than those of inherent metal ion compounds. For example, transition metal ion compounds such as manganese salts, nickel salts, iron salts, copper salts, and zinc salts; calcium salts, magnesium salts, and sodium salts are improved in their solubility in water when associated. Therefore, α-glycosyl α,α-trehalose can be advantageously used as a solubility-improving agent for those metal ion compounds. Further, associated complexes of those metal ion compounds and α-glycosyl α,α-trehalose can be advantageously used in the fields of foods, cosmetics, and pharmaceuticals, requiring the provision of the solution comprising metal ion compounds in high concentrations.

In the case of hardly soluble or insoluble substances, formed by associating metal ion compounds with organic substances except for saccharides such as glycosides and polyphenols, the solubility in water of the organic substances can be improved by associating the metal ion compounds with α-glycosyl α,α-trehalose. By forming associated complexes of metal ion compounds and α-glycosyl α,α-trehalose, the dirt formed by metal ion compounds can be prevented and easily removed by washing. Therefore, α-glycosyl α,α-trehalose can be advantageously used as a preventing agent, removing agent, washing agent, and bed-bath agent for pollution by metal ion compounds. Those agents can be preferably used for preventing or removing pollution of surfaces on glasses, metals, cars, houses, clothes, and bodies. Also, in the case of tartars or dental plaques resulting from calcium ion compounds and magnesium ion compounds, their adhesion can be inhibited and their dissolution can be promoted by associating the metal ion compounds with α-glycosyl α,α-trehalose. Therefore, α-glycosyl α,α-trehalose can be advantageously used for mouth wash and toothpaste.

(4) Inhibition of Oxidative or Reductive Action

Ionic compounds of transition metals such as iron and copper, and other metals may be oxidized or reduced depending on conditions. It is possible that such oxidative and reductive reactions deteriorate other substances which are coexisted with such ions. When such metal ion compounds form associated complexes with α-glycosyl α,α-trehalose, their oxidative or reductive activities are usually decreased. Therefore, associated complexes of metal ion compounds, which have potency of being oxidized or reduced, such as iron and copper salts; and α-glycosyl α,α-trehalose can be advantageously used as metal ion compound preparations without deteriorating qualities of other substances. By the properties, the oxidation and deterioration of substances, which are easily oxidized or deteriorated by the coexistence of a relatively low amount of iron salts or copper salts, for example, vitamins such as L-ascorbic acid and tocopherols, highly unsaturated fatty acids such as eicosapentaenoic acid (EPA) and docosahexanoeic acid (DHA), flavors, and colorings, can be inhibited by admixing with α-glycosyl α,α-trehalose to form associated complexes. Further, α-glycosyl α,α-trehalose inhibits the oxidation and reduction of ferrous ion, so that it can be advantageously used as a rust-preventing agent.

(5) Reduction of Unpleasant Tastes

Metal ion compounds, including salts such as magnesium chloride, sodium chloride, and ferrous chloride, may provide one unpleasant tastes such as bitterness, irritating taste, and metallic taste depending on an amount when one ingests them orally. Their unpleasant tastes can be reduced by forming associated complexes of metal ion compounds and α-glycosyl α,α-trehalose. Therefore, the associated complexes of α-glycosyl α,α-trehalose and metal ion compounds with unpleasant tastes according to the present invention are useful for producing foods and beverages, whose unpleasant tastes are reduced, in the field of foods. α-Glycosyl α,α-trehalose can be advantageously used as an unpleasant taste-reducing agent.

The associated complexes, formed by the method of the present invention, can be collected in a powdery form (in addition to liquid- and paste-forms) by the methods such as crystallization, fractional precipitation, concentration, and drying (including spray drying, drying in vacuo, and freeze-drying). The powdery associated complexes thus obtained have satisfactory features that undesirable properties such as deliquescent property, reducing power, oxidizing powder, and low solubility in water for industrial handling are improved in comparison with conventional powder preparations of metal ion compounds.

The powdery associated complexes of the present invention, which exhibit the effects described above, are very useful in various fields which use metal ion compounds as materials, ingredients, and products, for example, foods (including beverages), agricultural and marine products, cosmetics, pharmaceuticals, commodities, chemical industries, and productions of materials and ingredients. They can be used in an isolated form and optionally in a composition form along with other ingredients, for example, one or more fillers and excipients such as calcium carbonate, calcium phosphate, lactose, sugar alcohols, cyclic saccharides, dextrin, starch, and cellulose. Other ingredients, which can be used along with the associated complexes of the present invention in the form of a composition, are desirably biologically acceptable ingredients in the case of using such composition to living bodies. In the case of using in the field of foods, the associated complexes of the present invention can be used along with, for example, sweeteners such as sucrose, glucose, maltose, L-fucose, L-rhamnose, stevia, Glycyrrhiza glabla, L-aspartyl L-phenylalanine methyl ester, glycyrrhizinate, and sucralose; acidifiers such as adipic acid, citric acid, glucono delta lactone, acetic acid, tartaric acid, fumaric acid, and lactic acid; seasonings such as sodium aspartate, alanine, citric acid, glutamic acid, theanine, and sodium chloride; one or more colorings, flavors, reinforcement, swelling agents, preservatives, disinfectants, oxidation-preventing agents, decolorant, paste agents, stabilizing agents, and emulsifiers, which are generally used in foods.

Also, the associated complexes and compositions comprising the same of the present invention can be used for foods; concrete examples of which are various seasonings such as a table salt, soy sauce, powdered soy sauce, “miso (bean paste), “funmatsu-miso (a powdered miso), moromi (a refined sake), “hishio (a refined soy sauce), “furikake (a seasoned fish meal), mayonnaise, dressing, vinegar, “sanbai-zu (a sauce of sugar, soy sauce and vinegar), “funmatsu-sushi-zu (powdered vinegar for sushi), “chuka-no-moto (an instant mix for Chinese dish), “tentsuyu (a sauce for Japanese deep fat fried food), “mentsuyu (a sauce for Japanese vermicelli), bouillon, sauce, ketchup, “yakiniku-no-tare (a sauce for Japanese grilled meat), curry roux, instant stew mix, instant soup mix, “dashi-no-moto (an instant stock mix), nucleic acid seasoning, mixed seasoning, “mirin (a sweet sake), “shin-mirin (a synthetic mirin), table sugar, and coffee sugar; various “wagashi (Japanese cakes) such as “senbei (a rice cracker), “arare (a rice cake cube), “okoshi (a millet and rice cake), “mochi (a rise paste) and the like, “manju (a bun with a bean-jam), “uiro (a sweet rice jelly), Mann (a bean-jam) and the like, “yokan (a sweet jelly of beans), “mizu-yokan (a soft azuki-bean jelly), “kingyoku (a kind of yokan), jelly, pao de Castella, and “amedama (a Japanese toffee); western confectioneries such as a bun, biscuit, cracker, cookie, pie, pudding, butter cream, custard cream, cream puff, waffle, sponge cake, doughnut, chocolate, chewing gum, caramel, and candy; frozen desserts such as an ice cream and sherbet; syrups such as a “kajitsu-no-syrup-zuke (a preserved fruit) and “korimitsu (a sugar syrup for shaved ice); pastes such as a flour paste, peanut paste, fruit paste, and spread; processed fruits and vegetables such as a jam, marmalade, “syrup-zuke (fruit pickles), and “toka (conserves); pickles and pickled products such as a “fukujin-zuke (red colored radish pickles), “bettara-zuke (a kind of whole fresh radish pickles), “senmai-zuke (a kind of sliced fresh radish pickles), and “rakkyo-zuke (pickled shallots); premix for pickles and pickled products such as a “takuan-zuke-no-moto (a premix for pickled radish), and “hakusai-zuke-no-moto (a premix for fresh white rape pickles); meat products such as a ham and sausage; products of fish meat such as a fish ham, fish sausage, “kamaboko (a steamed fish paste), “chikuwa (a kind of fish paste), and “tempura (a Japanese deep-fat fried fish paste); dried marine products such as a dried seaweed, dried whole fish, opened and dried fish; “chinmi (relish) such as a “uni (urchin), “ika-no-shiokara (salted guts of squid), “su-konbu (processed tangle), “saki-surume (dried squid strips), “fugu-no-mirin-boshi (a dried mirin-seasoned swellfish); “tsukudani (foods boiled down in soy sauce) such as those of layer, edible wild plants, dried squid, small fish, and shellfish; daily dishes such as a “nimame (cooked beans), potato salad, and “konbu-maki (a tangle roll); milk products such as yoghurt and cheese; canned and bottled products such as those of fish meat, meat, fruit, and vegetable; alcoholic beverages such as a sake, synthetic sake, liqueur, and western liquor; soft drinks such as a coffee, tea, cocoa, juice, isotonic drink, mineral-supplement drink, mineral-enriched drink, carbonated beverage, fruit juice beverage, sour milk beverage, beverage containing a lactic acid bacterium, vegetable juice, and soymilk; instant food products such as instant pudding mix, instant hot cake mix, “sokuseki-shiruko (an instant mix of azuki-bean soup with rice cake), and instant soup mix; solid foods for babies, foods for therapy; health drinks such as a ginseng extract, bamboo leaf extract, plum extract, pine leaf extract, turtle extract, chlorella extract, aloe extract, propolis extract; and other foods and beverages such as peptide foods, frozen foods, healthy foods, viable cell of lactic acid bacteria and yeast, and royal jelly; mineral enrichments comprising calcium and/or magnesium, growth-promoting agent for Bacillus natto, flavor-improving agent, flavor-improving agent for soymilk, and coagulating agent for the production of “tofu (a bean curd).

In the case of using the associated complexes of the present invention in the field of agricultural and marine products, the associated complexes comprising minerals can be advantageously used intact or in a composition form comprising other additional ingredients for feeds and pet foods for animals or nutritional supplements or activating agents for plants. One or more ingredients, which are generally used in the following respective fields, for example, feeds or feed ingredients such as bagasse, corncob, rice straw, hay, grain, wheat flour, starch, oil meal, wasted sugar, wheat bran, bean cake, various fermentation cake, chip, and leaf and the like; and ingredients for nutritional supplements such as nitrate, ammonium salts, urea, phosphate, and potassium salts can be used as other ingredients which can be added to the composition.

Also, they can be advantageously used in various materials for concentrated feeds, feed mixtures, and pet food mixtures for domestic creatures, for example, domestic animals, poultry, honeybees, silkworms, insects, and fishes; and nutrition-supplements and activating agents for plants, for example, crops such as grains and potatoes and the like, vegetables, tea plants, fruit trees, planting of garden and roadside trees, and grasses of golf course.

In the case of using the associated complexes of the present invention in a composition form in the fields of cosmetics and pharmaceuticals, one or more of the following ingredients can be used conventionally in those fields, for example, moisture-retaining agents, detergents, colorings, flavors, enzymes, hormones, vitamins, ultraviolet rays (UV)-absorbing agents, UV-shielding agents, solvents, stabilizing agents, plasticizers, suaving agents, solubilizing agents, reducing agents, buffers, sweeteners, bases, vaporization-assisting agents, adsorbents, corrigents, synergists, binders, suspending agents, anti-oxidation agents, brightening agents, coating agents, dampers, refrigerants, softeners, emulsifiers, excipients, antiseptic agents, and preservatives.

Also, they can be advantageously used for specific products, for example, cosmetics such as a milky lotion, cream, shampoo, rinse, treatment, lipstick, rouge, lip cream, lotion, bath agent, and tooth paste; favorite products such as a tobacco and cigarette; pharmaceuticals such as an internal liquid medicine, tablet, slave, troche, cod-liver oil in the form of drop, oral refrigerant, cachou, gargle (mouthwash), magnesium supplement, and mineral-enrichment; and stabilizing agents for various enzymes.

In order to produce the compositions described above, the associated complexes of the present invention are preferably added to the compositions in the range of, usually, 0.00001-75% (w/w), desirably, 0.0001-50% (w/w), more desirably, 0.001-25% (w/w), on a dry solid basis (d.s.b.).

Using α-maltosyl α,α-trehalose as an α-glycosyl α,α-trehalose, the following Experiments 1 to 3 explain the fact that α-maltosyl α,α-trehalose forms associated complexes with various metal ion compounds. Experiments 4 to 7 explain the usefulness of the associated complexes.

Experiment 1

Preparation of α-maltosyl α,α-trehalose

Maltotetraose (purity: 97.9%), commercialized by Hayashibara Biochemical Laboratories Inc., Okayama, Japan, was prepared into 40% (w/w) aqueous solution (2,500 g) and the pH of the solution was adjusted to 7.0 with 1 M sodium hydroxide aqueous solution, and then pre-incubated at 40° C. A non-reducing saccharide-forming enzyme, which had been prepared from Arthrobacter sp. Q36 according to the method described in Japanese Patent Kokai No. 143,876/95, was admixed with the above pre-incubated solution to give an enzyme amount of four units/g-dry solid and subjected to an enzymatic reaction at a pH 7.0 and 40° C. for 38 hours. The reaction was stopped by heating to about 98° C. and keeping the temperature for 15 minutes. The α-maltosyl α,α-trehalose content of the reaction mixture was about 74.3%, d.s.b., by a sugar composition analysis using high performance liquid chromatography (abbreviated as “HPLC”, hereinafter). Reducing saccharides remaining in the reaction mixture were decomposed under an alkaline condition by the steps of adjusting the pH of the reaction mixture to about 12.5 by adding and dissolving sodium hydroxide in a granule form and keeping the temperature at about 98° C. and the pH at about 12.5 by gradually adding sodium hydroxide in a granule form. After cooling the resulting reaction mixture, it was desalted using an ion-exchange resin and decolored and filtrated using activated charcoal by the conventional method. The filtrate was further filtrated using a membrane with a pore size of 0.45 μm. The resulting filtrate was concentrated with an evaporator and dried in vacuo and to obtain about 779 grams of a powdery saccharide. The purity of α-maltosyl α,α-trehalose and the moisture content of the saccharide were respectively 98.7% and 7.18%. HPLC was carried out using “MCI GEL CK04SS” (internal diameter: 10 mm, length: 200 mm), a column commercialized by Mitsubishi Chemical Corporation, Tokyo, Japan, with connecting two columns, at a column temperature of 80° C. and a flow rate of 0.4 ml/minute of water, and using “R1-8012”, a differential refractometer commercialized by Tosoh Corporation, Tokyo, Japan.

Experiment 2

Associated Complex of α-maltosyl α,α-trehalose and Calcium Chloride

Experiment 2-1

Crystalline Associated Complex of α-maltosyl α,α-trehalose and Calcium Chloride

Experiment 2-1(a)

Isolation of Crystalline Associated Complex

14.5 grams (0.1 mole) of calcium chloride dihydrate were placed in a 200-ml glass beaker, admixed with 45 grams of deionized water, and dissolved completely by heating. Under continuous heating condition, 72.7 grams (0.1 mole) of a powdery α-maltosyl α,α-trehalose, prepared by the method in Experiment 1, were added to the solution and dissolved completely. After stopping the heating and preserving the beaker at room temperature (about 25° C.) for four days, precipitates comprising crystals were observed in the bottom of the beaker. The crystals were transferred to a bucket-type centrifugal separator and washed by spraying thereto an appropriate amount of water, followed by collecting the resulting crystals. The collected crystals were dried in vacuo at 40° C. for four hours. Further, the crystals were dried well by preserving in a desiccator containing phosphorous pentaoxide at room temperature for 20 hours. As a result, about 20 grams of a white crystalline powder were obtained.

Experiment 2-1(b)

Physicochemical Properties of Crystalline Associated Complex

(1) X-Ray Diffraction Analysis

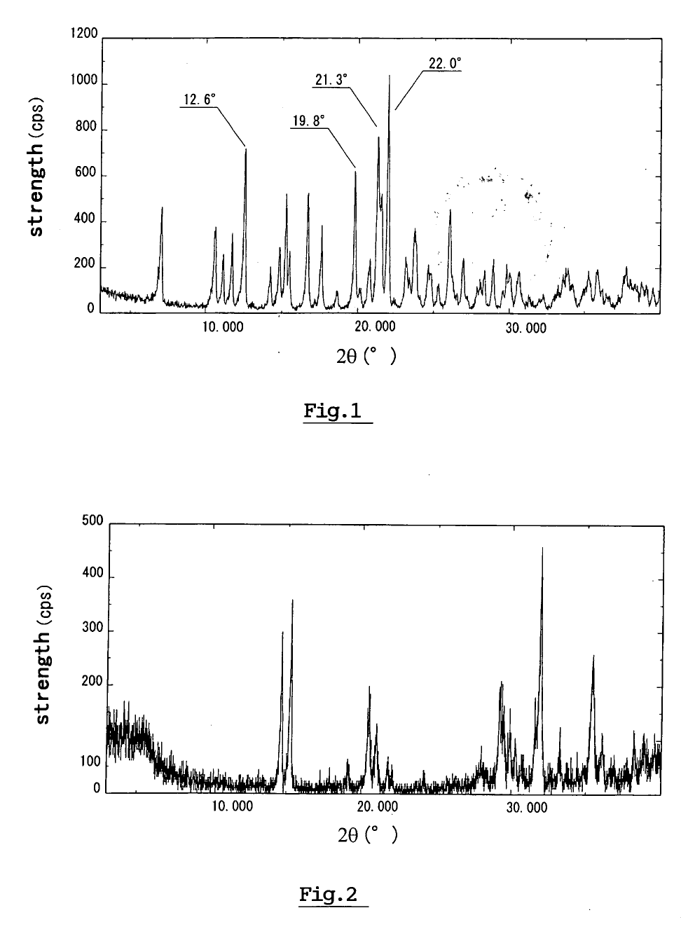

The crystal, obtained by the method described in Experiment 2-1 (a), was analyzed for X-ray diffraction pattern on conventional powdery X-ray diffraction analysis using an X-ray diffractometer, “RAD-2B”, commercialized by Rigaku Corporation, Tokyo, Japan. Also, X-ray diffraction pattern of calcium chloride dihydrate was analyzed with the same method. X-ray diffraction pattern of the crystalline associated complex obtained by the method in Experiment 2-1(a) and that of calcium chloride are respectively shown in FIGS. 1 and 2. As is evident from the results of FIGS. 1 and 2, X-ray diffraction pattern of FIG. 1 shows main characteristic diffraction angles (2θ) of 12.6°, 19.8°, 21.3°, and 22.0°, and it is quite different from that of calcium chloride shown in FIG. 2. Taking account of the fact that crystal of α-maltosyl α,α-trehalose has not been known yet and the results of the component analysis described below, the crystal obtained in Experiment 2-1(a) is quite different from crystalline calcium chloride dehydrate and has an independent crystallographic structure.

(2) Component Analysis

The crystal obtained by the method in Experiment 2-1(a) was analyzed for components as follows:

α-Maltosyl α,α-trehalose

Twenty-five milligrams of the crystal were dissolved in 5 ml of pyridine containing 2 mg/ml of phenyl-β-D-glucoside as an internal standard for gas chromatography. After converting the saccharide in 250 μl portion of each solution into a trimethylsiliy-derivative by the conventional method, the sample was analyzed on gas chromatography (a column, “OV-17, commercialized by GL Sciences, Inc., Tokyo, Japan). Separately, α-maltosyl α,α-trehalose prepared by the method in Experiment 1 as a standard was weighed accurately and analyzed by gas chromatography. The amount of α-maltosyl α,α-trehalose per one gram of the crystal was calculated based on the peak areas of the sample and the standard.

Calcium

Twenty-five milligrams of the crystal were dissolved in 1% (v/v) of hydrochloric acid, and the resulting solution was diluted to 100-fold with 10% (w/v) lanthanum chloride solution. Then, the calcium content of the diluted solution was measured using an atomic absorption photometry (“model 5100” commercialized by Perkin-Elmer Japan Co., Ltd., Yokohama, Japan). The amount of calcium chloride per one gram of crystal was calculated from the absorption on the basis of the hypothesis that all calcium comprised in crystal is in the form of calcium chloride.

Moisture

The amount of moisture per one gram of the crystal was measured by conventional loss-on-drying method using five grams of the crystal.

The results obtained from the above analyses are summarized in Table 1.

TABLE 1

| Amount (mg/g-crystal) | | | Molar ratio in the crystal | | |
|---|---|---|---|---|---|
| α-Maltosyl | Calcium | Mois- | α-Maltosyl | Calcium | Mois- |
| α,α-trehalose | Chloride* | ture | α,α-trehalose | Chloride* | ture |
| 771.6 | 130.9 | 97.5 | 1 | 1 | 5 |

*Amount of calcium chloride per 1 gram of crystal calculated based on the hypothesis that all calcium detected by atomic absorption photometry is in the form of calcium chloride.

From the results in Table 1, it was revealed that the crystal, obtained by the method in Experiment 2-1 (a), contained α-maltosyl α,α-trehalose, calcium chloride, and water in a molar ratio of 1:1:5 and was in the form of crystalline pentahydrate. Since any crystal of α-maltosyl α,α-trehalose has not been known, the saccharide has been recognized as that would not be crystallized. It was revealed that α-maltosyl α,α-trehalose can be crystallized by forming an associated complex with calcium chloride.

Experiment 2-2

NMR Analysis of an Associated Complex of α-maltosyl α,α-trehalose and Calcium Chloride

In order to analyze the mechanism of association of α-maltosyl α,α-trehalose and calcium chloride in crystalline associated complex of α-maltosyl α,α-trehalose and calcium chloride, the following NMR analysis was carried out:

(¹³C-NMR)

Fifty milligrams of the crystalline associated complex of α-maltosyl α,α-trehalose and calcium chloride, prepared by the method in Experiment 2-1, and α-maltosyl α,α-trehalose, prepared by the method in Experiment 1, were respectively dissolved in one milliliter of 99.9% deuterium oxide, and analyzed on ¹³C-NMR as follows. NMR analysis was done using an instrument, model JNM-AL300”, commercialized by JOEL Ltd., Tokyo, Japan, a nucleus measurement of ¹³C, and resonance frequency of 75.45 MHz. After setting a tube containing the above solution to the instrument, spin-lattice relaxation time (herein after, called simply relaxation time”) of individual carbon atoms of α-maltosyl α,α-trehalose in the solution was measured according to the inversion recovery method described in the operation manual attached to the instrument. Individual peak (chemical shift, ppm), obtained as the result of analysis, was assigned based on the data described by J. H. Bradbery et al. in Carbohydrate Research, Vol. 126, 125-126, (1984) and described on the basis of carbon atom Nos. of α-maltosyl α,α-trehalose shown in Chemical formula 1. Assignments of carbon atoms and their relaxation times are in Table 2. (The result of α-maltosyl α,α-trehalose only and the associated complex of α-maltosyl α,α-trehalose and calcium chloride are in Tables 2-1 and 2-2, respectively.)

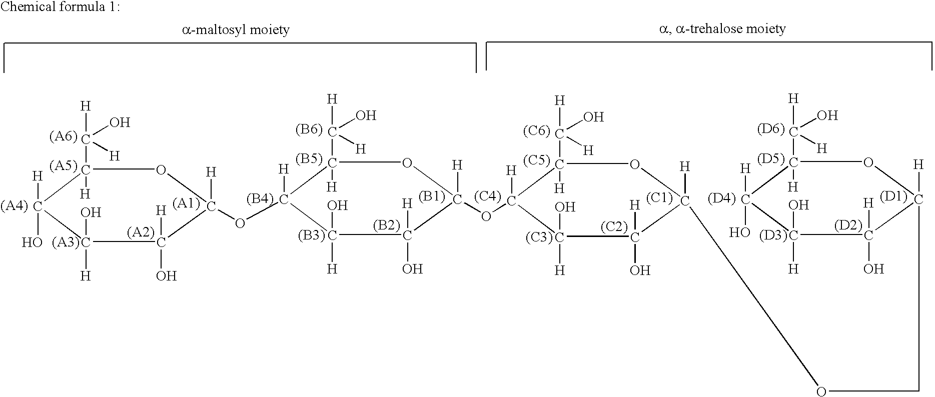

TABLE 2
1. α-Maltosyl α,α-trehalose

| Assignment | Chemical Shift | Relaxation Time |
|---|---|---|
| (Carbon atom No.) | (ppm) | (msec) |
| A-1 | 102.395 | 216 |
| A-2 | 74.810 | 193 |
| A-3 | 75.510 | 217 |
| A-4 | 71.958 | 164 |
| A-5 | 73.466 | 143 |
| A-6 | 63.213 | 137 |
| B-1 | 102.189 | 125 |
| B-2 | 74.381 | 178 |
| B-3 | 75.964 | 206 |
| B-4 | 79.384 | 177 |
| B-5 | 74.167 | 150 |
| B-6 | 63.123 | 137 |
| C-1 | 95.801 | 164 |
| C-2 | 73.821 | 189 |
| C-3 | 75.609 | 225 |
| C-4 | 79.821 | 140 |
| C-5 | 73.301 | 182 |
| C-6 | 63.123 | 137 |
| D-1 | 96.032 | 226 |
| D-2 | 73.705 | 160 |
| D-3 | 75.337 | 159 |
| D-4 | 72.304 | 157 |
| D-5 | 75.189 | 199 |
| D-6 | 63.123 | 137 |

2. Associated complex of α-maltosyl α,α-trehalose
and calcium chloride

| Assignment | Chemical Shift | Relaxation Time | |
|---|---|---|---|
| (Carbon atom No.) | (ppm) | (msec) | (%)* |
| A-1 | 102.411 | 197 | 91 |
| A-2 | 74.826 | 199 | 103 |
| A-3 | 75.543 | 171 | 79 |
| A-4 | 73.491 | 185 | 80 |
| A-5 | 72.008 | 146 | 102 |
| A-6 | 63.172 | 115 | 84 |
| B-1 | 102.213 | 147 | 117 |
| B-2 | 74.414 | 192 | 108 |
| B-3 | 75.998 | 209 | 101 |
| B-4 | 79.441 | 94 | 53 |
| B-5 | 74.200 | 134 | 89 |
| B-6 | 63.172 | 115 | 84 |
| C-1 | 95.818 | 177 | 108 |
| C-2 | 73.854 | 141 | 75 |
| C-3 | 75.626 | 151 | 67 |
| C-4 | 79.862 | 146 | 104 |
| C-5 | 73.334 | 179 | 98 |
| C-6 | 63.172 | 115 | 84 |
| D-1 | 96.057 | 208 | 92 |
| D-2 | 73.722 | 184 | 115 |
| D-3 | 75.354 | 170 | 107 |
| D-4 | 72.337 | 156 | 98 |
| D-5 | 75.214 | 201 | 101 |
| D-6 | 63.172 | 115 | 84 |

*Relative relaxation time, when the corresponding relaxation time of α-maltosyl α, α-trehalose is regarded as 100%. Bold letter means that the relaxation time of the carbon atom, reduced remarkably by the association.

As shown in Table 2, the relaxation times of carbon atoms at B4, C2 and C3 positions were remarkably reduced in the associated complex of α-maltosyl α,α-trehalose and calcium chloride. Therefore, it was suggested that the direct interaction between carbon atoms at B-4, C-2 and C-3 positions and calcium chloride involved in the association of α-maltosyl α,α-trehalose and calcium chloride. Different from the case of α,α-trehalose described in International Publication No. WO 03/016325, the α-maltosyl moiety (carbon atom at B4 position) of α-maltosyl α,α-trehalose is unexpectedly involved in the formation of the associated complex, as well as the α,α-trehalose moiety.

Experiment 3

Associated Complexes of α-maltosyl α,α-trehalose and Other Metal Ion Compounds

Experiment 3-1

NMR Analysis of Associated Complex of α-maltosyl α,α-trehalose and Magnesium Chloride or Strontium Chloride

A mixture, composed of 2.03 grams of magnesium chloride hexahydrate and 7.27 grams of α-maltosyl α,α-trehalose prepared by the method in Experiment 1 (molar ratio of 1:1), was admixed with four grams of deionized water and dissolved completely by heating. Similarly, a mixture, composed of 2.66 grams of strontium chloride hexahydrate and 7.27 grams of α-maltosyl α,α-trehalose prepared by the method in Experiment 1 (molar ratio of 1:1), was admixed with four grams of deionized water and dissolved completely by heating. After cooling each solution to ambient temperature, they were dried in vacuo at 80° C. for 15 hours. The resulting dried materials were pulverized conventionally to produce two kinds of powders.

(¹³C-NMR)

According to the method in Experiment 2-2, 50 mg of either of the above two kinds of powders were respectively dissolved in one ml of deuterium oxide, and the relaxation times of respective carbon atoms of α-maltosyl α,α-trehalose were analyzed on ¹³C-NMR. The relative values of the relaxation times of respective carbon atoms, thus obtained, to the relaxation times of respective carbon atoms obtained from α-maltosyl α,α-trehalose only were calculated based on the results of α-maltosyl α,α-trehalose obtained in Experiment 2-2 in Table 2-1. The results are summarized in Table 3.

TABLE 3

| | | |
|---|---|---|
| Assignment | Chemical Shift | Relaxation Time |

| | | | |
|---|---|---|---|
| (Carbon atom No.) | (ppm) | (msec) | (%)* |

1. Associated complex of α-maltosyl α,α-trehalose
and magnesium chloride

| | | | |
|---|---|---|---|
| A-1 | 102.386 | 179 | 83 |
| A-2 | 74.818 | 193 | 100 |
| A-3 | 75.527 | 193 | 89 |
| A-4 | 71.991 | 141 | 86 |
| A-5 | 73.483 | 169 | 118 |
| A-6 | 63.156 | 114 | 83 |
| B-1 | 102.189 | 139 | 111 |
| B-2 | 74.389 | 151 | 85 |
| B-3 | 75.972 | 157 | 76 |
| B-4 | 79.417 | 156 | 88 |
| B-5 | 74.183 | 132 | 88 |
| B-6 | 63.156 | 139 | 83 |
| C-1 | 95.801 | 195 | 119 |
| C-2 | 73.829 | 155 | 82 |
| C-3 | 75.617 | 103 | 46 |
| C-4 | 79.829 | 139 | 99 |
| C-5 | 73.310 | 157 | 86 |
| C-6 | 63.156 | 139 | 83 |
| D-1 | 96.032 | 129 | 57 |
| D-2 | 73.714 | 136 | 85 |
| D-3 | 75.337 | 132 | 83 |
| D-4 | 72.321 | 136 | 85 |
| D-5 | 75.197 | 211 | 106 |
| D-6 | 63.156 | 139 | 83 |

2. Associated complex of α-maltosyl α,α-trehalose
and strontium chloride

| | | | |
|---|---|---|---|
| A-1 | 102.444 | 223 | 103 |
| A-2 | 74.867 | 220 | 114 |
| A-3 | 75.543 | 130 | 60 |
| A-4 | 72.040 | 175 | 107 |
| A-5 | 73.524 | 182 | 127 |
| A-6 | 63.189 | 141 | 103 |
| B-1 | 102.238 | 135 | 108 |
| B-2 | 74.439 | 186 | 105 |
| B-3 | 76.021 | 217 | 105 |
| B-4 | 79.466 | 136 | 77 |
| B-5 | 74.233 | 174 | 116 |
| B-6 | 63.189 | 141 | 103 |
| C-1 | 95.859 | 182 | 111 |
| C-2 | 73.878 | 224 | 119 |
| C-3 | 75.667 | 169 | 75 |
| C-4 | 79.878 | 149 | 106 |
| C-5 | 73.359 | 193 | 106 |
| C-6 | 63.189 | 141 | 103 |
| D-1 | 96.090 | 203 | 90 |
| D-2 | 73.755 | 181 | 113 |
| D-3 | 75.387 | 170 | 107 |
| D-4 | 72.370 | 136 | 103 |
| D-5 | 75.238 | 211 | 117 |
| D-6 | 63.156 | 139 | 103 |

*Relative relaxation time, when the corresponding relaxation time of α-maltosyl α,α-trehalose is regarded as 100%.
Bold letter means that the relaxation time of the carbon atom, reduced remarkably by the association.

As is evident from the results in Table 3, both the two kinds of powders, obtained from the mixture of α-maltosyl α,α-trehalose and magnesium chloride and that of α-maltosyl α,α-trehalose and strontium chloride, by dissolving and drying in vacuo, showed remarkably reduced relaxation times of carbon atoms at specific positions in comparison with the case of α-maltosyl α,α-trehalose only. From the results, it was revealed that α-maltosyl α,α-trehalose formed an associated complex with magnesium chloride or strontium chloride by direct interaction as in the case of calcium chloride, i.e., the above two kinds of powders were respectively associated complexes of α-maltosyl α,α-trehalose and magnesium chloride, and of α-maltosyl α,α-trehalose and strontium chloride. Further, from the results in Table 3-1, it was supposed that the interaction of carbon atoms at B-3, C-3 and D-1 positions of α-maltosyl α,α-trehalose and metal ion compounds was involved deeply in the formation of an associated complex of α-maltosyl α,α-trehalose and magnesium chloride. Also, from the results in Tables 3-2, it was supposed that the interaction of carbon atoms at A-3, B-4 and C-3 positions of α-maltosyl α,α-trehalose and metal ion compounds was involved deeply in the formation of an associated complex of α-maltosyl α,α-trehalose and strontium chloride. These results indicate that the α-maltosyl moiety of α-maltosyl α,α-trehalose is involved in the formation of the associated complex, as well as the α,α-trehalose moiety, as in the case of an associated complex of α-maltosyl α,α-trehalose and calcium chloride.

Experiment 3-2

Change of Solubility of Metal Ion Compounds by the Association with α-maltosyl α,α-trehalose

The changes of solubility in water of metal ion compounds were examined under coexistence of α-maltosyl α,α-trehalose and metal ion compounds. α-Maltosyl α,α-trehalose prepared by the method in Experiment 1 was used for the tests. Strontium chloride hexahydrate, cuprous chloride dihydrate, ferrous chloride tetrahydrate, manganese chloride tetrahydrate, and nickel chloride hexahydrate were used as metal ion compounds for the tests. 72.7 grams of α-maltosyl α,α-trehalose (0.1 mole) and 0.1 mole of either of the above metal ion compounds were placed in a 100 ml-glass beaker and admixed with deionized water to give 30 grams of water per beaker with taking account of the moisture content of α-maltosyl α,α-trehalose and the bound water of metal ion compounds, and then the contents in the beaker were dissolved by heating. Solutions prepared by dissolving the same amount of metal ion compounds only were prepared as controls. After dissolving the contents completely, all the beakers were allowed to stand at room temperature (25° C.) for 24 hours. Successively, precipitation of crystals was judged by macroscopic observation. In the case of precipitating crystals, the formed crystals were collected and analyzed for components by the conventional method. The results are in Table 4.

TABLE 4

| | Precipitation of crystal | |
|---|---|---|
| | In the presence of | In the absence of |
| | α-maltosyl | α-maltosyl |
| Metal ion compound | α,α-trehalose | α,α-trehalose |
| Strontium chloride | Crystalline strontium | Precipitated |
| | chloride was slightly | (strontium chloride) |
| | precipitated. | |
| Cuprous chloride | Crystalline cuprous | Precipitated |
| | chloride was slightly | (cuprous chloride) |
| | precipitated. | |
| Ferrous chloride | Crystalline ferrous | Precipitated |
| | chloride was slightly | (ferrous chloride) |
| | precipitated. | |
| Manganese chloride | None | Precipitated |
| | | (manganese chloride) |
| Nickel chloride | None | Precipitated |
| | | (nickel chloride) |

Referring to the case of strontium chloride (Table 4, the first row), which was confirmed to form an associated complex with α-maltosyl α,α-trehalose by NMR analysis (Experiment 3-1), crystals of strontium chloride were clearly observed in the absence of α-maltosyl α,α-trehalose. However, the amount of crystals was remarkably decreased in the presence of α-maltosyl α,α-trehalose. Referring to the results of cuprous chloride, ferrous chloride, manganese chloride, and nickel chloride (Table 4, from the second row through the fifth row), the solubility in water of these metal ion compounds were remarkably improved in the presence of α-maltosyl α,α-trehalose as in the case of strontium chloride. Accordingly, based on the above judgement, it was suggested that these metal ion compounds also formed associated complexes with α-maltosyl α,α-trehalose in the presence of α-maltosyl α,α-trehalose.

Experiment 4

Hygroscopicity of Associated Complex of α-maltosyl α,α-trehalose and Calcium Chloride

In order to compare the hygroscopicity (deliquescent property) of an associated complex of α-maltosyl α,α-trehalose and calcium chloride with that of a control, calcium chloride which is known to have a high deliquescent property, the following hygroscopicity test was carried out. The crystalline associated complexes of α-maltosyl α,α-trehalose and calcium chloride, prepared according to the method in Experiment 2-1 and calcium chloride dehydrate, were used as test samples. The moisture content (weight of moisture per one gram of the sample) of each sample was measured by conventional drying loss method. The moisture contents of an associated complex of α-maltosyl α,α-trehalose and calcium chloride, and calcium chloride dihydrate were 0.097 g and 0.245 g, respectively. These samples, about 1.5 g each, were respectively placed in an aluminum cup and preserved at 25° C. for seven days in a desiccator which was controlled to keep at a relative humidity of 52.8%. The weight of the content in each cup was measured at the initiation of preservation (0 day preservation), and 1, 2, 4, and 7 days after the initiation. The amount of moisture per one gram of each sample after 1 to 7 days preservation was calculated based on the assumption that the increased amount measured to that of 0 day preservation was the amount of moisture absorbed by each sample. The results are in Table 5.

TABLE 5

| Relative | | Moisture content/g-sample (g) | | | | |
|---|---|---|---|---|---|---|
| Humidity | Sample* | 0 day | 1 day | 2 days | 3 days | 7 days |
| 52.8% | CaCl₂•2H₂O | 0.245 | 0.319 | 0.359 | 0.405 | 0.430 |
| | MT*-CaCl₂ | 0.097 | 0.188 | 0.188 | 0.186 | 0.188 |

*Denotation of “MT” means α-maltosyl α,α-trehalose.

As shown in Table 5, calcium chloride dihydrate began to absorb moisture from the beginning of the preservation under the relative humidity of 52.8%. The moisture content per one gram of the sample was reached 0.430 gram after 7 days preservation. On the other hand, the associated complex of α-maltosyl α,α-trehalose and calcium chloride showed almost no moisture absorption through one to seven days preservation even though it showed a slight increase in the moisture content after one day preservation. The degree of absorbing moisture of the sample was evidently low in comparison with the case of calcium chloride. Further, the associated complex of α-maltosyl α,α-trehalose and calcium chloride showed no deliquescence under the macroscopic observation whereas calcium chloride showed that. The above results indicate that the inherent property for the deliquescence of calcium chloride is remarkably improved in the associated complex of α-maltosyl α,α-trehalose and calcium chloride.

Experiment 5

Inhibiting Effect of α-maltosyl α,α-trehalose on the Formation of Calcium Phosphate Precipitate

When phosphate ion is added to a calcium chloride aqueous solution, calcium phosphate, an insoluble salt, is formed from calcium ion and phosphate ion and then precipitated. Effects of α-maltosyl α,α-trehalose and other saccharides on the phenomenon were investigated as follows. A calcium chloride aqueous solution was prepared by the steps of adding 3.68 grams of calcium chloride dihydrate to deionized water to dissolve therein and adding deionized water to the solution to give a total volume of 200 ml. α-Maltosyl α,α-trehalose, prepared by the method in Experiment 1, maltotetraose, and anhydrous crystalline sucrose were used as saccharides for the test. A phosphate solution (pH 6.8) was prepared by the steps of mixing 250 ml of 0.2 M potassium dihydrogen phosphate solution with 118 ml of 0.2 M sodium hydroxide solution, and filling up the resulting solution to one liter with deionized water.

Twenty-six grams, d.s.b., of either of the saccharides for the test were added to five milliliters of the above calcium chloride solution and dissolved by adding additional deionized water. The resulting aqueous solutions were filled up to give a total volume of 50 ml. A control solution was prepared by the steps of adding deionized water only to five ml of the above calcium chloride solution and filling up the resulting solution to give a total volume of 50 ml with deionized water. Successively, 40 ml of the above phosphate solution was mixed with 10 ml of calcium chloride solution. After stirring at 37° C. for three hours, each solution was centrifuged at 10,000 rpm for 10 minutes and the resulting supernatant was collected. The calcium concentration (the solubilized calcium concentration) of each supernatant was measured using “ZEEMAN 5100”, an atomic adsorption-photometer commercialized by Perkin-Elmar Japan Co. Ltd., Kanagawa, Japan. Samples for measurement were prepared by the steps of adding two ml of 10% (w/v) lanthanum chloride solution to five ml of each of the above supernatants after centrifugation and filling up the resulting solution to give 25 ml with deionized water.

The above procedure was carried out for individual each sample (three kinds of saccharides and control) for three times and the average value of the solubilized calcium concentration was calculated. The results are summarized in Table 6.

TABLE 6

| | Concentration of solubilized calcium |
|---|---|
| Saccharide | (mg/L, average ± standard deviation) |
| None (Control) | 6.86 ± 0.49 |
| α-Maltosyl α,α-trehalose | 9.86 ± 0.53 |
| Maltotetraose | 6.91 ± 0.83 |
| Sucrose | 6.54 ± 0.31 |

As shown in Table 6, in the case of using α-maltosyl α,α-trehalose only, the concentration of solubilized calcium was remarkably high in comparison with the cases of other saccharides. Therefore, it was revealed that α-maltosyl α,α-trehalose had an ability of inhibiting the precipitation of calcium phosphate which is formed by the coexistence of calcium ion and phosphate ion. From the results in Experiments 2 and 3 showing that α-maltosyl α,α-trehalose formed an associated complexes with metal ion compounds, it was considered that α-maltosyl α,α-trehalose inhibited the formation of insoluble salt (calcium phosphate), formed via the ionic bond between calcium ion and phosphate ion, by associating with the solubilized calcium salt (calcium chloride in this experiment).

Experiment 6

Inhibiting Effect of α-maltosyl α,α-trehalose on the Oxidation of Ferrous Ion

Generally, ferrous ion (divalent, Fe²⁺) and ferric ion (trivalent, Fe³⁺) are known as ions of iron element. Ferrous ion is easily oxidized with light or heat and converted to ferric ion. Effects of α-maltosyl α,α-trehalose on the phenomenon were investigated as follows. An aqueous solution, comprising ferrous chloride (FeCl₂) tetrahydrate corresponding to the amount of 1% (w/v) as ferrous ion, and comprising α-maltosyl α,α-trehalose corresponding to the amount of 5% (w/v), was prepared as a test solution. While, a solution, comprising ferrous chloride only with the same concentration to the test solution, was prepared as a control solution. After preparing the test and control solutions, small portions of which were respectively sampled, and the amounts of ferrous ion were measured by the Nitroso-DMAP method described later. Successively, the test and control solutions, 10 ml each, were respectively placed in a 20 ml-vial and sealed. After preserving these vials at 37° C. for four hours while irradiating a light with about 9,000 luxes, the amount of ferrous ion in each solution was measured by the Nitroso-DMAP method. Measurement by the Nitroso-DMAP method was carried out as follows. After diluting the test or control solution accurately by 100-fold, 0.5 ml of the dilute was placed in a 50 ml-volumetric flask. Successively, five ml of 0.2% (w/v) nitroso-dimethyl-aminophenol in 0.1 N hydrochloric acid solution and four ml of 3 N ammonium buffer (pH8.5) were added quickly to the dilute and filled up accurately to give a volume of 50 ml with deionized water. After the above procedure, the absorbance at 750 nm (within the visible light range) of the solution was measured. The standard solutions, prepared by stepwisely diluting a ferrous chloride solution with known concentration, were used for measurement in the same manner as above to obtain a standard curve for quantitative analysis. The amount of ferrous ion of the test or the control solution was determined with the standard curve. The results are in Table 7.

TABLE 7

Amount of Fe²⁺ ion (mg/ml)

| | Before | After |
|---|---|---|
| Saccharide | light-irradiation | light-irradiation |
| α-Maltosyl | 10.2 | 7.4 |
| α,α-trehalose | | |
| None | 10.2 | 3.8 |
| (Control) | | |

As shown in Table 7, in the test solution comprising α-maltosyl α,α-trehalose, ferrous ion was remained in an evidently large amount after light-irradiation in comparison with that for the control solution. Taking account of the results and those in Experiment 3-2 showing the formation of an associated complex of α-maltosyl α,α-trehalose and an iron salt, it is considered that the above inhibiting effect by α-maltosyl α,α-trehalose is the result of forming an associated complex of the saccharide and an iron salt.

Experiment 7

Inhibiting Effect of α-maltosyl α,α-trehalose on the Deterioration of Ascorbic Acid Under the Presence of Metal Ion

L-Ascorbic acid deteriorates rapidly by the oxidative degradation in the presence of iron and copper ions and causes browning. The effects of α-maltosyl α,α-trehalose on the phenomenon were investigated as follows. Five kinds of aqueous solutions, having respective compositions described in Table 8, were prepared. Aqueous solutions of L-ascorbic acid alone or L-ascorbic acid and metal ion compounds were used as controls. The test solutions were prepared by adding α-maltosyl α,α-trehalose to the control solutions. Ten milliliters of each of these test and control solutions were placed in different 20 ml-vials and sealed. The vials were preserved at 50° C. The control and test solutions, containing ferrous chloride, and those containing ferric chloride or copper sulfate were respectively preserved for 96 hours and 40 hours. After the preservation, the degree of coloring of each solution was measured. In the case of the control solutions containing L-ascorbic acid alone, the degrees of coloring were measured at the preservation periods of 40 hours and 96 hours. The absorbance at 420 nm (within the visible light range) of each sample was measured for the degree of coloring. The results are shown in Table 8.

TABLE 8

| | | Degree of coloring | |
|---|---|---|---|
| | | (Abs.** at 420 nm) | |
| | | Preserved | Preserved |
| Composition of solution* | Note | for 40 hrs | for 96 hrs |
| 14.8 mM AsA | Control | 0.012 | 0.166 |
| 14.8 mM AsA + 10 mM CuSO₄ | Control | 0.800 | — |
| 14.8 mM AsA + 10 mM CuSO₄ + | Test | 0.332 | — |
| 100 mM MT | | | |
| 14.8 mM AsA + 10 mM FeCl₃ | Control | — | 0.588 |
| 14.8 mM AsA + 10 mM FeCl₃ + | Test | — | 0.225 |
| 100 mM MT | | | |

*Denotations of “AsA” and “MT” mean L-ascorbic acid and α-maltosyl α,α-trehalose, respectively;
**Absorbance

As shown in Table 8, degree of coloring of the test solutions, containing α-maltosyl α,α-trehalose, was remarkably low in comparison with that of the control. Taking account of the results and those in Experiment 3-2, which showed the formation of an associated complex of α-maltosyl α,α-trehalose and an iron salt or a copper salt, it is considered that the above inhibiting effects by α-maltosyl α,α-trehalose are the results of forming an associated complex of the saccharide and iron or a copper salt.

The following examples explain associated complexes of the present invention and their uses in detail.

### EXAMPLE 1

Associated Complex of α-maltosyl α,α-trehalose and Calcium Chloride

According to the method in Experiment 2-1, a crystalline associated complex of α-maltosyl α,α-trehalose and calcium chloride was prepared. Since the associated complex has an improved deliquescence in comparison with calcium chloride, it has a satisfactory handleability on preserving or admixing with various compositions.

Further, since the above associated complex hardly forms an insoluble salt, calcium phosphate, when admixed with a composition comprising phosphoric acid, phosphate salt, or phosphate ion; products, which are prevented from getting cloudy or being precipitated, can be obtained by using the associated complex as materials of aqueous solutions with calcium, such as isotonic drinks, nutritional supplements, and external preparations for the skin. Therefore, the crystalline associated complex of α-maltosyl α,α-trehalose and calcium chloride is very useful as a material of products comprising calcium in various fields of foods, cosmetics, pharmaceuticals, etc.

### EXAMPLE 2

Associated Complexes of α-maltosyl α,α-trehalose and Various Metal Ion Compounds

One part by weight of α-maltosyl α,α-trehalose, prepared according to the method in Example 1, and equimolars with the α-maltosyl α,α-trehalose of either of magnesium chloride hexahydrate, strontium chloride hexahydrate, ferrous chloride tetrahydrate, cupric chloride tetrahydrate, nickel chloride hexahydrate, or manganese chloride hexahydrate were mixed, admixed with 0.53 part by weight of deionized water, and dissolved completely by heating. After cooling, the resulting each solution was dried in vacuo at 80° C. for 15 hours. Six kinds of powdery associated complexes were obtained by pulverizing the resulting dried matters.

Since these associated complexes have an improved solubility in water in comparison with metal ion compounds alone, products, which are prevented from getting cloudy or being precipitated, can be obtained by using the associated complexes as materials of aqueous solutions with metal ion compounds, such as isotonic drinks, nutritional supplements, and external preparation for skin. Therefore, the associated complexes are very useful as a material of products comprising metal ion compounds in various fields of foods, cosmetics, pharmaceuticals, etc.

### EXAMPLE 3

Powdery Isotonic Drink

According to the formula described below, a powdery composition was prepared by mixing respective ingredient sufficiently.

| | | |
|---|---|---|
| Hydrous crystalline maltose | 6,000 | parts by weight |
| Sucrose | 5,000 | parts by weight |
| Vitamin B₁ | 0.1 | part by weight |
| Vitamin B₂ | 0.3 | part by weight |
| Vitamin B₆ | 0.4 | part by weight |
| Vitamin C | 200 | parts by weight |
| Niacin | 4 | parts by weight |
| Disodium phosphate (anhydrate) | 93 | parts by weight |
| Potassium dihydrogen phosphate (anhydrate) | 62 | parts by weight |
| Associated complex of α-maltosyl | 90 | parts by weight |
| α,α-trehalose and magnesium chloride, | | |
| prepared by the method in Example 2 | | |
| Associated complex of α-maltosyl | 55 | parts by weight |
| α,α-trehalose and calcium chloride, | | |
| prepared by the method in Example 1 | | |

The above powdery composition was divided into 200 ml-plastic bottles with screw caps to produce powdery isotonic drinks. The product can be drunk after admixing 10 grams of the product with about 100 ml of water and dissolving the admixture. Since the associated complexes of α-maltosyl α,α-trehalose and metal ion compounds, comprised in the product, have low deliquescence properties, the product can be preserved for a long period of time. Also, the associated complexes comprised in the product are convenient because they are rapidly dissolved in water. Further, since the associated complex of α-maltosyl α,α-trehalose and calcium chloride, comprised in the product, hardly forms insoluble salts from phosphate ion and precipitates when dissolved in water, the product has a feature of hardly deteriorating the absorbability of each component when the product solution is drunk after dissolving and leaving for a relatively long period of time.

### EXAMPLE 4

Skin Lotion for External Use

According to the formula described below, a liquid composition was prepared by mixing and dissolving the ingredients.

| Citric acid | 0.02 | part by weight |
|---|---|---|
| Sodium citrate | 0.08 | part by weight |
| 1,3-Butylenglycol | 2 | parts by weight |
| Ethanol | 2 | parts by weight |
| Anhydrous crystalline maltitol | 1 | part by weight |
| Hydrous crystalline trehalose | 0.2 | part by weight |
| 2-O-α-D-glucosyl-L-ascorbic acid | 0.5 | part by weight |
| Associated complex of α-maltosyl | 0.0035 | part by weight |
| α,α-trehalose and ferrous chloride, | | |
| prepared by the method in Example 2 | | |
| Purified water | the rest of above | |
| Total | 100 | parts by weight |

The above liquid composition was divided into 100 ml-glass bottles with screw caps to produce skin lotion. Since the product provides adequate refreshing sense and moisture-retaining ability, it is useful as a basic skin care for keeping the health of the skin. Since the associated complex of α-maltosyl α,α-trehalose and ferrous chloride, comprised in the product, hardly causes the deterioration of other components, the prescribed effects can be obtained after preserving for a relatively long period of time.

### EXAMPLE 5

Vitamin Supplement

According to the formula described below, a powdery composition was prepared by mixing each ingredient sufficiently.

| | | |
|---|---|---|
| Folic acid | 0.0004 | part by weight |
| L-Ascorbic acid | 0.2 | part by weight |
| Associated complex of α-maltosyl | 5 | parts by weight |
| α,α-trehalose and magnesium chloride, | | |
| prepared by the method in Example 2 | | |
| Associated complex of α-maltosyl | 0.008 | part by weight |
| α,α-trehalose and manganese chloride, | | |
| prepared by the method in Example 2 | | |
| α-Maltosyl α,α-trehalose | 5 | parts by weight |

The above powdery composition was divided into 80 ml-glass bottles with screw caps to produce a vitamin supplement. The product is ingested about 10 grams per day as a rough standard and can be drunk after adding about 100 ml of water or hot water to 10 grams of the product and dissolving. Since associated complexes of α-maltosyl α,α-trehalose and metal ion compounds, comprised in the product, can be dissolved rapidly in water, the use of the product is very easy.

### EXAMPLE 6

Table Salt

According to the formula described below, solids were prepared by mixing each ingredient sufficiently and drying under a reduced pressure. The resulting solids were pulverized to prepare a powdery table salt.

| | |
|---|---|
| Sodium chloride | 90 parts by weight |
| Associated complex of α-maltosyl | 12 parts by weight |
| α, α-trehalose and magnesium chloride, | |
| prepared by the method in Example 9 | |

The table salt has a low hygroscopicity and a satisfactory fluidity. Since unpleasant tastes of bittern such as pungent taste and bitter taste, originated from sodium chloride and bittern components, were suppressed and the product has a good taste by harmonizing sodium chloride, α-maltosyl α,α-trehalose, and magnesium chloride adequately, the product can be used for cooking and seasoning of foods (including grilled foods) and beverages, and enjoyed the flavor of them. Also, since the product has a similar composition with seawater, it is a mild salt for living bodies. For example, an aqueous solution, comprising the product at about 3% concentration, can be advantageously used for removing sands from seashells.

### EXAMPLE 7

Processed Soymilk

Processed soymilk was produced according to the procedure described below. Ten parts by weight of material soybeans were removed the skins. After autoclaving beans at 130° C. for 10 minutes, 90 parts by weight of hot water was admixed with the beans and the beans were milled. About 60 parts by weight of soymilk was obtained by removing residues (bean curd refuse) from the mixture solution by the centrifugation. Ten parts by weight of a reduced syrup comprising α-glycosyl α,α-trehalose (which comprises, on a dry solid basis, about 4% of α-glucosyl α,α-trehalose, about 52% of α-maltosyl α,α-trehalose, and about 1% of α-maltotriosyl α,α-trehalose), and which is prepared by hydrogenating “HALLODEX®, a syrup commercialized by Hayashibara Shoji Inc., Okayama, Japan, five parts by weight of “SUNMALT®, a powdery crystalline maltose commercialized by Hayashibara Shoji Inc., Okayama, Japan, 0.05 part by weight of table salt prepared by the method in Example 6. 0.02 part by weight of soybean oil, and a suitable amount of lecithin were added to the soy milk and dissolved. A processed soy milk was produced by the steps of sterilizing the above mixture by heating, deodorizing in vacuo, admixing with a suitable amount of flavor, homogenizing, cooling, filling a vessel, and wrapping.

Different from the conventional similar soymilk, the processed soymilk comprises α-glucosyl α,α-trehalose, α-maltosyl α,α-trehalose, α-maltotriosyl α,α-trehalose, and a small amount of magnesium. Therefore, it is a quaffable beverage with a good feeling without bitterness, harsh taste, and scratchiness.

### EXAMPLE 8

“Tofu (Bean Curd)

A “tofu (bean curd) was produced according to the procedure described below. One part by weight of soybeans was washed with water, soaked in water for 12 hours, and then ground. After adding five parts by weight of water to the ground material and boiling for five minutes, the resulting mixture was filtered with a cloth to produce soymilk. A “tofu was produced by the steps of adding one part by weight each of pullulan and an associated complex of α-maltosyl α,α-trehalose and magnesium chloride, obtained by the method in Example 2, as a coagulating agent at 70° C. to one hundred parts by weight of the soymilk, and coagulating it.

A processing efficiency in the production of the present “tofu is improved because the time which is required to coagulate the soymilk is prolonged to about seven minutes in comparison with the case of using a bittern. Since the “tofu comprises pullulan and α-maltosyl α,α-trehalose, it shows a low syneresis and a high yield and has a satisfactory texture, gloss, and flavor. The product has a satisfactory preservability and can be used to cook chilled “tofu cut into cubes, boiled “tofu, “miso soup.

### EXAMPLE 9

“An (Sweetened Bean Paste)

One hundred parts by weight of raw “an prepared from “azuki beans by a usual manner, 60 parts by weight of sucrose, 14.5 parts by weight of “HALLODEX®, a syrup commercialized by Hayashibara Shoji Inc., Okayama, Japan, which comprises, on a dry solid basis, about 4% of α-glucosyl α,α-trehalose, about 52% of α-maltosyl α,α-trehalose, and about 1% of α-maltotriosyl α,α-trehalose, and one part by weight of an associated complex of α-maltosyl α,α-trehalose prepared by the method in Example 2 were admixed with seven parts by weight of water and dissolved by heating with gentle stirring. The mixture was boiled to give a Brix value of 56 to make-into “an.

Although the product comprises higher amount of magnesium in comparison with the usual “an, it has a low bitterness and viscosity. The “an has a satisfactory taste and flavor and does not cause the deterioration of color. The product can be preferably used as “an material for bread and Japanese confectioneries such as “manju (Japanese bean cake), “dango (Japanese rice cake), “monaka (a Japanese cake), and “hyoka.

### EXAMPLE 10

Feed Mixture

According to the formula described below, a feed mixture was prepared by mixing each ingredient.

| | | |
|---|---|---|
| Powdery gluten | 40 | parts by weight |
| Skim milk | 38 | parts by weight |
| Lactosucrose | 12 | parts by weight |
| Vitamin preparation | 10 | parts by weight |
| Fish meal | 5 | parts by weight |
| Calcium diphosphate | 5 | parts by weight |
| Liquid fat | 3 | parts by weight |
| Calcium carbonate | 3 | parts by weight |
| Sodium chloride | 2 | parts by weight |
| Associated complex of α-maltosyl | 2 | parts by weight |
| α,α-trehalose and calcium chloride, | | |
| prepared by the method in Example 1 | | |

The above feed mixture is hardly denatured because an associated complex of α-maltosyl α,α-trehalose and calcium chloride, which is admixed as minerals with the composition, shows no deliquescence. The product has an improved preference for domestic animals and poultry, especially, for pig. The product has a function of promoting the growth of Bifidobacteria and can be advantageously used for preventing infection and diarrhea of domestic animals, promoting appetite and fattening of them, and suppressing smell of their excrement. The product can be optionally mixed with other feed materials, for example, grains, wheat flour, starch, oil cake, and molasses to make rich feeds. Also, the product can be used along with crude feed materials such as straws, hey, bagasse, and corncob to make into other feed mixtures.

### EXAMPLE 11

Cosmetic Cream

According to the formula described below, ingredients were mixed and processed to produce a cosmetic cream. Two parts by weight of polyoxyethylenglycol monostearate, five parts by weight of self-emulsified glycerin monostearate, two parts by weight of “αG-HESPERIDINE®, α-glucosyl hesperidin commercialized by Hayashibara Shoji Inc., Okayama, Japan, one part by weight of liquid paraffin, 10 parts by weight of glycerin trioctanoate, and a suitable amount of preservative were mixed and dissolved by heating in a usual manner. Two parts by weight of sodium L-lactate, five parts by weight of 1,3-butylene glycol, two parts by weight of an associated complex of α-maltosyl α,α-trehalose and magnesium chloride, prepared by the method in Example 22, and 66 parts by weight of deionized water were admixed with the above mixture and emulsified using a homogenizer. The resulting mixture was further admixed with suitable amount of perfume, and stirred to produce a cosmetic cream. The product has a moisture-retaining property because it comprises an associated complex of α-maltosyl α,α-trehalose and magnesium chloride, and is useful as a sunburn preventive, skin-care agent, and whitening agent.

### EXAMPLE 12

Salve (External Preparation)

A salve (external preparation) was prepared by mixing ingredients according to the formula described below. Two hundred parts by weight of a powder comprising an associated complex of α-maltosyl α,α-trehalose and magnesium chloride, prepared by the method in Example 2, 300 parts by weight of maltose, and 50 parts by weight of methanol comprising three parts by weight of iodine were mixed. Further, 200 parts by weight of an aqueous solution containing pullulan in an amount of 10% (w/w) was admixed with the above mixture to produce a salve for curing wounds, which has a adequate spread property and adherability.

Since the product is prepared by using an associated complex of α-maltosyl α,α-trehalose and magnesium chloride, the working efficiency for the production is improved. In addition to the disinfectant activity of iodine, α-maltosyl α,α-trehalose comprised in the product can be used as an energy-supplementing agent for cells. Therefore, the use of the product enables to shorten the curing period and to cure wounds prettily.

### EXAMPLE 13

Nutritional Supplement for Plants

A nutritional supplement for plants in liquid form was prepared by mixing the ingredients according to the formula described below.

| | | |
|---|---|---|
| Diammonium phosphate | 132 | parts by weight |
| Ammonium nitrate | 17.5 | parts by weight |
| Potassium chloride | 71.5 | parts by weight |
| Associated complex of α-maltosyl | 360 | parts by weight |
| α,α-trehalose and magnesium chloride, | | |
| prepared by the method in Example 2 | | |
| Water | 1,000 | parts by weight |

The product comprises nitrogen (N), phosphate (P₂O₅), potassium (K₂O), and magnesium (MgO) in a ratio of 10:20:15:3, respectively. The product has activities of promoting the growth of plants and the running of roots when plants are rooted or replanted, and improving the blooming flowers and the bearing of fruits. The product can be used by properly diluting with water as a nutritional supplement for plants such as crops including grains and potatoes, vegetables, fruit trees, garden plants, trees of garden and roadside, and grasses in golf courses.

### EXAMPLE 14

Bath Agent

A bath agent was prepared by mixing the ingredients according to the formula described below.

| | | |
|---|---|---|
| Sodium bicarbonate | 80 | parts by weight |
| Dried sodium sulfate | 12 | parts by weight |
| Potassium chloride | 4 | parts by weight |
| Sedimentary calcium carbonate | 2 | parts by weight |
| α-Maltosyl α,α-trehalose | 50 | parts by weight |
| “αG-HESPERIDINE ®, α-glucosyl hesperidine | 2 | parts by weight |
| Associated complex of α-maltosyl | 100 | parts by weight |
| α,α-trehalose and magnesium chloride, | | |
| obtained by the method in Example 2 | | |

| | |
|---|---|
| Colorings and flavors | proper amount |

Since the product comprises α-maltosyl α,α-trehalose and magnesium, it has a satisfactory moisture-retaining property and heat-retentive property and is suitable as skin-care agents and whitening agents. The product can be used by diluting 1,000 to 10,000-fold with warm water for taking a bath. The product has a merit of decreasing soap-sediments, scales. Further, the product can be also used as cleansing lotions and lotions by diluting it.

### EXAMPLE 15

Mineral Water

A mineral water was prepared according to the procedure described below. α-Maltosyl α,α-trehalose was added to ground water which was pumped up in a mountain to give the concentration of 0.5% (w/w) and dissolved. The resulting solution was sterilized by filtering with a membrane filter. Sterilized bottles were filled with the filtrate to produce bottled mineral waters. The contents of major metal ion compounds in the mineral water were 40.9 ppm of calcium, 12.5 ppm of sodium, and 11.6 ppm of magnesium.

Since the product comprises α-maltosyl α,α-trehalose, associated complexes of α-maltosyl α,α-trehalose and metal ion compounds are formed. The product is not clouded during the preservation for a long period because the solubility of the associated complexes is improved. It is a high quality mineral water, comprising suitable amounts of minerals, with a good feeling, which gives no thirsty.

### EXAMPLE 16

Dried “Wakame (Brown Seaweed)

α-Maltosyl α,α-trehalose was added to seawater to give the concentration of 8% (w/w) and dissolved by heating. After blanching the “wakame in the solution whose temperature was kept to 80-85° C., it was dried to produce a dried “wakame.

Since α-maltosyl α,α-trehalose and bittern components (magnesium chloride, calcium chloride, etc.) comprised in seawater form associated complex on the surface of “wakame, the hygroscopicity of the product after drying was decreased, and the product shows no stickiness caused by the moisture-adsorption during the preservation. The product can be advantageously used as a material for salad. Further, the product is useful as foods such as confectionery and food materials.

### EXAMPLE 17

Toothpaste

Toothpaste was prepared by mixing the components according to the formula described below.

| | | |
|---|---|---|
| Calcium phosphate (CaHPO₄) | 45 | parts by weight |
| Sodium lauryl sulfate | 1.5 | parts by weight |
| Glycerin | 25 | parts by weight |
| Polyoxyethylenesorbitan laurate | 0.5 | parts by weight |
| α-Maltosyl α,α-trehalose, | 20 | parts by weight |
| obtained by the method in Experiment 1 | | |
| Preservatives | 0.05 | part by weight |
| Water | 13 | parts by weight |

The product is improved in unpleasant taste and has a satisfactory availability without decreasing the washing power of detergents. Since α-glycosyl α,α-trehalose, which are comprised in the product, form associated complexes with metal ion compounds, the product has abilities of inhibiting the adhesion of tartars and dental plaques, which are formed by calcium and magnesium ion compounds, and promoting the dissolution of them. Therefore, the product has a satisfactory tooth-brushing power.

### INDUSTRIAL APPLICABILITY

As described above, the present invention revealed that α-glycosyl α,α-trehalose formed associated complexes with metal ion compounds by direct interactions in the presence of metal ion compounds. Since the associated complexes of the present invention have the improved deliquescence, the high solubility in water, and the decreased reactivity against the oxidation and reduction, they are very useful in their industrial handleability in comparison with conventional metal ion compounds. The associated complexes of the present invention can be advantageously used in various fields which use metal ion compounds as materials, ingredients, and products, for example, foods (including beverages), agricultural and marine products, cosmetics, pharmaceuticals, commodities, chemical industries, and industries for producing materials or ingredients, which are used in their fields.

The present invention, having these outstanding functions and effects, is a significantly important invention that greatly contributes to this art.

1. An associated complex comprising α-glycosyl α,α-trehalose and a metal ion compound.

2. The associated complex of claim 1, wherein said metal ion compound is one or more compounds selected from metal salts.

3. The associated complex of claim 1, wherein said metal ion compound is one or more inorganic salts.

4. The associated complex of claim 3, wherein said inorganic salt comprises a divalent or greater metal cation, and one or more inorganic anions as anion.

5. The associated complex of claim 3, wherein said inorganic salt comprises calcium ion.

6. The associated complex of claim to 5, wherein said inorganic salt is calcium chloride.

7. The associated complex of claim 3, wherein the molar ratio of said inorganic salt to said α-glycosyl α,α-trehalose is about one.

8. The associated complex of claim 3, wherein said inorganic salt comprises one or more cations selected from the group consisting of magnesium ion, strontium ion, copper ion, ferrous ion, manganese ion, and nickel ion.

9. A crystalline associated complex of claim 1.

10. The crystalline associated complex of claim 9, which is formed from α-glycosyl α,α-trehalose and calcium chloride.

11. The crystalline associated complex of claim 10, wherein the molar ratio of said calcium chloride to said α-glycosyl α,α-trehalose is about one.

12. A crystalline associated complex of α-glycosyl α,α-trehalose and calcium chloride, which has main diffraction angles (2θ) of 12.6°, 19.8°, 21.3°, and 22.0° on powdery X-ray diffraction analysis.

13. A process for producing an associated complex of α-glycosyl α,α-trehalose and a metal ion compound, which comprises the steps of:
forming an associated complex of α-glycosyl α,α-trehalose and a metal ion compound by mixing said α-glycosyl α,α-trehalose with said metal ion compound; and
collecting the resulting associated complex.

14. The process of claim 13, wherein said α-glycosyl α,α-trehalose and said metal ion compound are mixed in a solution.

15. The process of claim 13, wherein said metal ion compound is calcium salt.

16. The process of claim 15, wherein said calcium salt is calcium chloride.

17. The process of claim 13, which comprises the steps of:
crystallizing said associated complex from a solution; and
collecting the resulting crystal.

18. A method for forming an associated complex of α-glycosyl α,α-trehalose and a metal ion compound by mixing said α-glycosyl α,α-trehalose with said metal ion compound.

19. The method of claim 18, wherein said α-glycosyl α,α-trehalose and said metal ion compound are mixed in a solution.

20. The method of claim 18, wherein said metal ion compound is calcium salt.

21. The method of claim 20, wherein said calcium salt is calcium chloride.

22. A composition comprising the associated complex of claim 1 together with another component.

23. A composition comprising the crystalline associated complex of claim 9.

24. The composition of claim 22, which is a food, cosmetic, or pharmaceutical.

25. The composition of claim 22, wherein said composition is a member selected from the group consisting of mineral-extender, mineral-supplying agent, taste-improving agent, bean curd (tofu)-coagulating agent, moisture-retaining agent,

26. An agent comprising α-glycosyl α,α-trehalose as an effective ingredient, selected from the group consisting of an unpleasant-taste suppressing agent, deliquescence-suppressing agent, crystallization-suppressing agent for hardly water-soluble or water-insoluble substance, adhesion-suppressing agent, solubility-improving agent, cleaning agent, bed-bath agent, and oxidation-reduction suppressing agent, for metal ion compounds or products comprising the same.
